(19) United States
(12) Patent Application Publication
(10) Pub. No.: US 2013/0204147 A1
(43) Pub. Date: Aug. 8, 2013

(54) Atrial Fibrillation Detection Based On Pulmonary Artery Pressure Data

(71) Applicants: Andreas Blomqvist, Valencia, CA (US); Michael E. Benser, Valencia, CA (US)

(72) Inventors: Andreas Blomqvist, Valencia, CA (US); Michael E. Benser, Valencia, CA (US)

(73) Assignee: PACESETTER, INC., Sylmar, CA (US)

(21) Appl. No.: 13/366,106

(22) Filed: Feb. 3, 2012

(51) Int. Cl. A61B 5/0215; A61B 5/021

(52) U.S. Cl. 600/486; 600/485

(57) ABSTRACT

Atrial fibrillation (AF) is detected based on pulmonary artery pressure (PAP) data. In some embodiments, PAP data generated by a PAP sensor device implanted in or near the pulmonary artery of a patient is processed to determine whether the patient is suffering from AF. In some aspects, detection of AF is based on identifying cycle-to-cycle variations of one or more parameters derived from the PAP data.

### TECHNICAL FIELD

This application relates generally to atrial fibrillation detection and more specifically, but not exclusively, to detecting atrial fibrillation based on pulmonary artery pressure data.

### BACKGROUND

Stroke is a leading cause of death in the United States and treatment costs (e.g., hospitalization) associated with stroke patients are significant. The risk of stroke more than doubles each decade after the age of 55. Accordingly, it is desirable to prevent stroke whenever possible.

A significant risk factor for thrombotic stroke is atrial fibrillation (AF), which may increase the risk of stroke five-fold. However, it is not uncommon that AF is first diagnosed in a patient after the patient has suffered a stroke (e.g., undiagnosed AF may be present in roughly 1 out of every 10 patients with ischemic stroke). Thus, in cases where AF is unrecognized in a patient, the AF may progress untreated with potentially dire consequences. Accordingly, it is desirable to detect AF in a patient before the patient suffers a stroke.

### SUMMARY

A summary of several sample aspects of the disclosure and embodiments of an apparatus constructed or a method practiced according to the teaching herein follows. It should be appreciated that this summary is provided for the convenience of the reader and does not wholly define the breadth of the disclosure. For convenience, one or more aspects or embodiments of the disclosure may be referred to herein simply as “some aspects” or “some embodiments.”

The disclosure relates in some aspects to detecting AF in a patient. In this way, appropriate treatment may be prescribed for the patient, thereby reducing the risk of stroke for the patient.

The disclosure relates in some aspects to detecting AF based on pulmonary artery pressure (PAP) monitoring. For example, a PAP sensor device implanted in or near the pulmonary artery of a patient may generate PAP data that is then processed to determine whether the patient is suffering from AF. In some aspects, detection of AF is based on identifying cycle-to-cycle variations of one or more parameters derived from the PAP data.

Various morphological characteristics of a PAP signal may be used individually or in combination to detect or confirm AF. Many of these characteristics are related to the temporal cycle-to-cycle stability of various measures, including peak-to-peak amplitude (Pmax−Pmin over the course of each cardiac cycle), morphological consistency (correlation of the PAP signal over each cardiac cycle to the prior cardiac cycle or a running average or some established template), systolic phase-to-systolic phase timing, as well as rate changes as assessed from the PAP signal. By these means, AF may be detected, and secondarily forms of AF burden may be determined.

Upon detecting an AF condition, an indication of AF may be communicated to the patient and/or a clinician for optimal management of the patient. In various embodiments, such an indication may be generated by an implantable device, an external monitor device, or some other external device (e.g., a remotely-located processing system).

The teachings herein may be employed for patients who do not already have an implantable medical device (IMD) or for patient who already have an IMD. In the former case, an implantable PAP sensor device may transmit PAP data or an indication of AF to an external monitor device. In the latter case, an implantable PAP sensor device may transmit PAP data or an indication of AF to the IMD and the IMD may, in turn, transmit the PAP data or an indication of AF to an external monitor device. In some cases, the PAP-based AF indication may be used to incrementally increase the accuracy of AF detection above and beyond that provided by the IMD.

### BRIEF DESCRIPTION OF THE DRAWINGS

These and other aspects of the disclosure will be more fully understood when considered with respect to the following detailed description, the appended claims, and the accompanying drawings, wherein:

FIG. 1 is a simplified block diagram of an embodiment of an apparatus for detecting atrial fibrillation;

FIG. 2 is a simplified flowchart of an embodiment of operations that may be performed to detect atrial fibrillation;

FIG. 3 is a simplified diagram of a PAP waveform and an IEGM waveform;

FIG. 4 is a simplified block diagram of an embodiment of a system for detecting atrial fibrillation where an implantable sensor device transmits PAP data or an AF indication to an external monitor device;

FIG. 5 is a simplified block diagram of an embodiment of a system for detecting atrial fibrillation where an implantable cardiac device transmits PAP data or an AF indication to an external monitor device;

FIG. 6 is a simplified block diagram of a system including a sample embodiment of an implantable sensor device and a sample embodiment of an external monitor device;

FIG. 7 is a simplified block diagram of a system including sample embodiments of an implantable sensor device, an external monitor device, and a processing system;

FIG. 8 is a simplified block diagram of a sample embodiment of an implantable sensor device;

FIG. 9 is a simplified block diagram of a system including a sample embodiment of an implantable sensor device and a sample embodiment of an implantable medical device;

FIG. 10 is a simplified flowchart of an embodiment of operations that may be performed to generate an indication of atrial fibrillation; and

FIG. 11 is a simplified flowchart of an embodiment of PAP-based AF detection operations that may be performed in conjunction with obtained IEGM data.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

AF is a conduction disorder that leads to irregular ventricular intervals. This stands in contrast to sinus rhythm where the ventricular intervals are very consistent. In accordance with the teachings herein, irregularity in the ventricular intervals may be manifested as irregularity in pressure waveforms in the pulmonary artery and these pressure waveforms may be analyzed to determine whether AF is indicated. In practice, such irregularities may be more difficult to detect in the pulmonary circulation as opposed to the systemic circulation since blood pressure changes are generally smaller here. However, in accordance with the teachings herein, a relatively small (and, in some cases, wireless) pressure sensor device is implanted directly within or near the pulmonary artery, thereby enabling accurate detection of PAP in a relatively minimally invasive manner. The pressure sensor device may sense (e.g., measure) PAP and generate PAP data (e.g., analog signals or digital data) that may then be used to detect AF in accordance with the teachings herein.

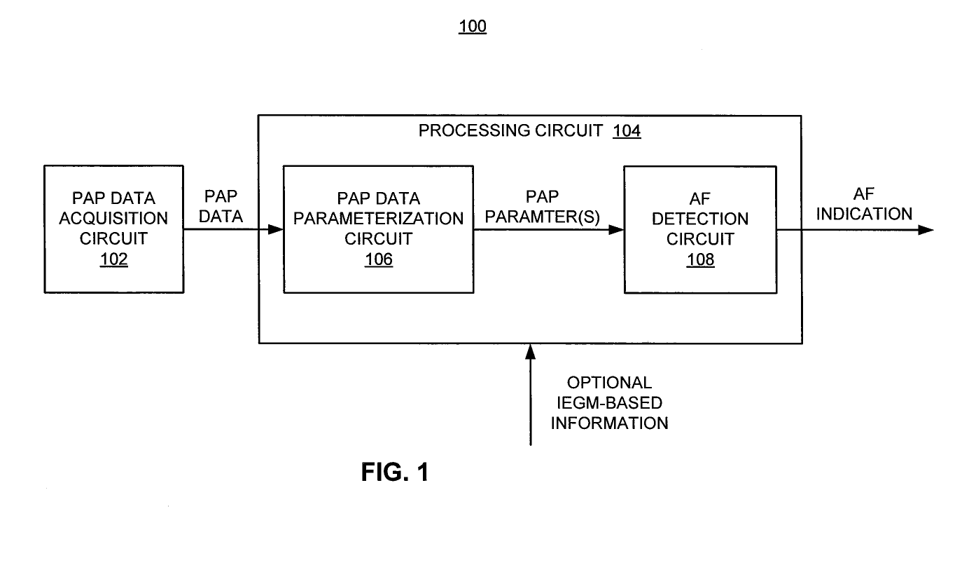

FIG. 1 illustrates an embodiment of an apparatus 100 for detecting AF. A PAP data acquisition circuit 102 obtains PAP data and provides the data to a processing circuit 104. As discussed in more detail below, in different embodiments, obtaining this PAP data may involve sensing PAP and generating the PAP data or receiving the PAP data from another apparatus.

The processing circuit 104 processes the PAP data to generate an AF indication. As illustrated in FIG. 1, in some embodiments, such a processing circuit comprises a PAP data parameterization circuit 106 and an AF detection circuit 108.

The PAP data parameterization circuit 106 processes the PAP data to generate parameters that are used to determine the cycle-to-cycle stability of the PAP over a series of cardiac cycles (i.e., cardiac beats). For example, the parameterization circuit 106 may identify certain characteristics of a PAP waveform represented by the PAP data. These characteristics may include, for example, the duration of each cycle, the amplitude of each cycle (e.g., peak-to-peak amplitude), frequency components of each cycle, and morphology correlation information for each cycle. For a given one of these characteristics, the parameterization circuit 106 generates a parameter that is indicative of how that characteristic varies over the series of cardiac cycles.

The AF detection circuit 108 determines whether to generate an indication of AF based on the PAP parameter(s) generated by the parameterization circuit 106. For example, in some embodiments, each PAP parameter is compared with a threshold and an indication of AF is generated based on this comparison.

In some embodiments, the processing circuit 104 also makes use of intracardiac electrogram-based information for AF detection. As discussed in more detail below, such intracardiac electrogram (IEGM) information may be provided, for example, by sensing circuitry of an implantable cardiac device. In some embodiments, IEGM information is provided to the parameterization circuit 104 to assist in identifying the beginning and end of cardiac cycles. In some embodiments, an indication of AF that is based on IEGM information is provided to the AF detection circuit 108 so that PAP-based AF detection may be used to confirm or reject IEGM-based AF detection, or vice-versa.

As discussed in more detail below, the apparatus 100 may take different forms in different embodiments. For example, the apparatus 100 may be implemented as or implemented in an implantable PAP sensor device, an implantable cardiac device, an external monitor device, an external processing device, or some other suitable device.

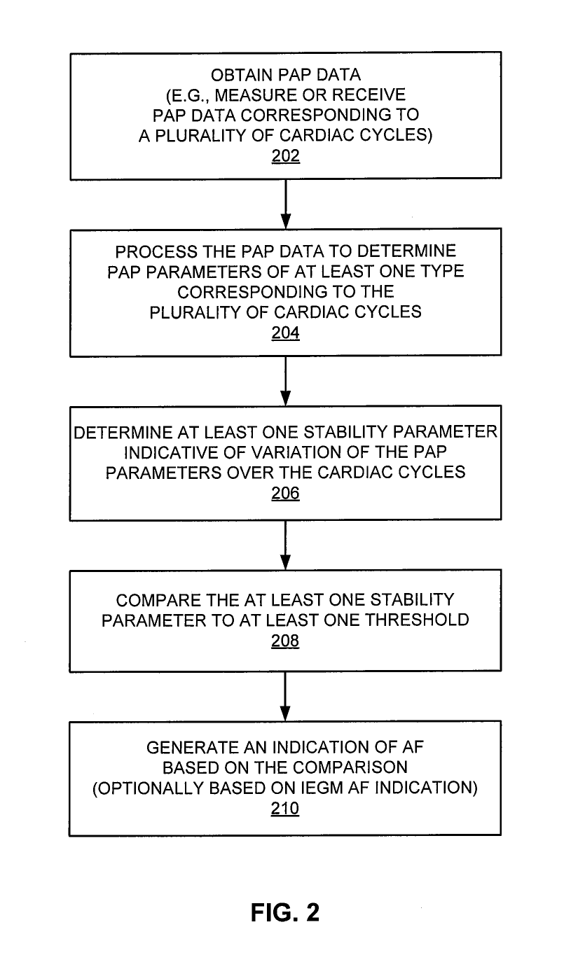

With the above overview in mind, sample AF detection operations will now be described in more detail in conjunction with the flowchart of FIG. 2. For convenience, the operations of FIG. 2 (or any other operations discussed or taught herein) may be described as being performed by specific components (e.g., components as described in FIG. 1 and FIGS. 4-9). It should be appreciated, however, that these operations may be performed by other types of components and may be performed using a different number of components. It also should be appreciated that one or more of the operations described herein may not be employed in a given implementation.

The operations of FIG. 2 are typically performed on a repeated basis to track a patient's condition over time. For example, PAP measurements may be conducted periodically (e.g., on a daily basis) in some embodiments. As another example, PAP measurements may be triggered by a defined event (e.g., IEGM-based alerts) in some embodiments. Each time this information is obtained, a decision may be made as to whether the patient is exhibiting signs of AF.

As represented by block 202, PAP data is obtained at some point in time. In general, this PAP data corresponds to several contiguous cardiac cycles (e.g., at least three cycles). For example, PAP may be measured by an implantable PAP sensor for a defined period of time (e.g., 30 seconds) to generate the PAP data.

As described in more detail below, the obtaining of the PAP data at block 202 involves different operations in different embodiments. In embodiments where the operations of FIG. 2 are performed in an implantable PAP sensor device, obtaining the PAP data may involve sensing (e.g., measuring) a PAP signal via a PAP sensor circuit. In embodiments where these operations are performed in an implantable medical device that communicates with an implantable PAP sensor device, obtaining the PAP data may involve receiving the PAP data (e.g., via signaling carried over an inter-device lead or via wireless signaling). In embodiments where these operations are performed in an external device (e.g., a monitor device) that communicates with an implantable PAP sensor device, obtaining the PAP data may involve receiving the PAP data via wireless signaling. In embodiments where these operations are performed in an external device that communicates with another external device (e.g., a monitor device), obtaining the PAP data may involve receiving the PAP data via a network connection.

As represented by block 204, the PAP data is processed (e.g., by a processing circuit) to determine PAP parameters of at least one type corresponding to the plurality of cardiac cycles. As discussed above, the different types of PAP parameters may include, for example, cycle-to-cycle timing parameters, per-cycle amplitude parameters, per-cycle morphology correlation parameters, and per-cycle frequency parameters.

A different number of parameter types may be employed in different embodiments. For example, some embodiments will only employ one parameter type (e.g., timing parameters will be obtained for the different cycles). In other embodiments, two parameters types will be employed (e.g., timing parameters and amplitude parameters will be obtained for the different cycles). Other embodiments may employ additional parameter types.

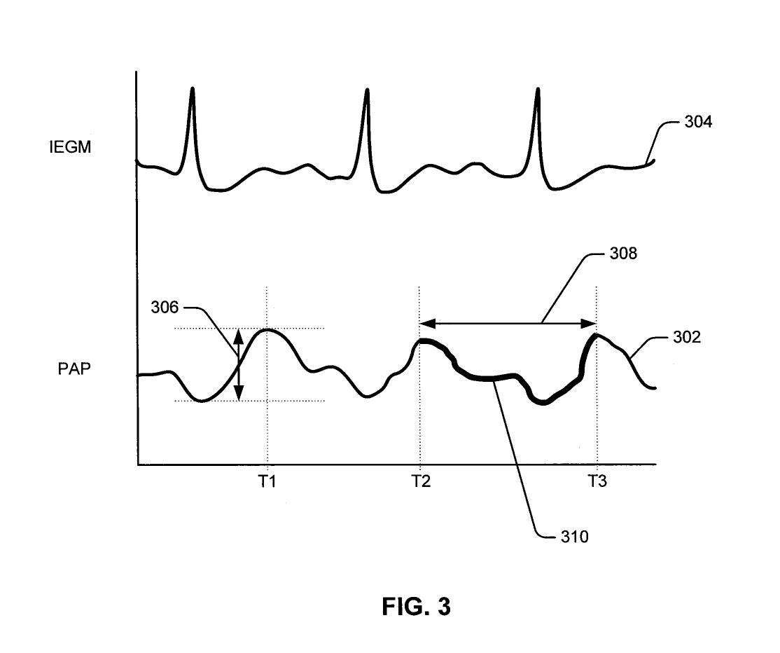

FIG. 3 illustrates a simplified example of a PAP waveform 302 that may be represented by PAP data. FIG. 3 also illustrates a corresponding IEGM waveform 304 (e.g., representative of cardiac signals sensed at the same time as the PAP).

Determining PAP parameters as discussed herein may thus involve identifying the different cycles of the PAP waveform 302. In a typical implementation, cycles are identified by comparing the waveform data to a threshold to identify zero crossings (e.g., in a positive direction) or some other characteristic that is indicative of different cycles. In embodiments where IEGM information is available, cycles may be identified based on the IEGM information.

The arrowed line 306 illustrates an example of a peak-to-peak amplitude parameter that may be calculated for a given cycle. Within a given cycle, the maximum and minimum values are identified and the difference calculated to find the peak-to-peak amplitude for that cycle. A similar operation is then performed for the other cycles of the PAP waveform 302.

The arrowed line 308 illustrates an example of a cycle-to-cycle timing parameter that may be calculated for two consecutive cycles. Similar timing parameters may be calculated for the other consecutive cycle pairs of the PAP waveform 302.

Cycle-to-cycle timing may be determined in various ways. For example, different cycle timing points (e.g., PAP waveform maximum, PAP waveform zero crossing, IEGM-specified event, etc.) may be used to calculate the time period of each cycle. Examples of cycle timing points based on PAP waveform maximums are designated by T1, T2, and T3 in FIG. 3. In some embodiments, the duration of each cycle is calculated directly. For example, a timer may be re-started at each cycle timing point. The value of the timer just before it is re-started may then be logged to determine the duration of that cycle. In other embodiments, timestamps (e.g., based on a running clock) may be generated for each cycle timing point (e.g., at T1, T2, T3, and so on). In these cases, the duration of each cycle is determined by determining the time differences between these timestamps.

The bolded section 310 of the waveform 302 illustrates an example of a morphology correlation parameter that may be calculated for a given cycle. For example, a set of data representative of incremental rises and falls of the waveform may be obtained for each cycle. In this way, information indicative of the different shapes of different cycles may be recorded.

In other embodiments, Fourier processing or some other suitable processing may be employed to determine one or more frequency characteristics associated with a given cycle. For example, a set of data representative of the corresponding frequency spectrum may be obtained for each cycle. In this way, information indicative of the different frequency components of different cycles may be recorded.

Referring again to FIG. 2, as represented by block 206, at least one parameter indicative of variation of the PAP parameters over the cardiac cycles is determined (e.g., by the processing circuit). This parameter may be referred to as a stability parameter in the discussion that follows. In some aspects, the stability parameter is defined such that it provides an indication as to how a particular parameter (e.g., cycle-to-cycle timing) changes on a cycle-to-cycle basis. Here, it will be appreciated that AF is indicated by irregular changes on a cycle-to-cycle basis, as opposed to sinus rhythm where such parameters are relative constant on a cycle-to-cycle basis. Several examples of how different parameters may change during AF follow.

Regarding timing parameters, AF will result in irregularities in the durations of the ventricular cycles. For example, one ventricular cycle may be significantly shorter (e.g., by 0.1 seconds) than a previous ventricular cycle. This irregularity in the durations of the ventricular cycles may be identified by tracking the durations of the PAP cycles. For example, in some embodiments, AF is indicated if the duration of one or more PAP cycles (e.g., maximum-to-maximum, zero crossing-to-zero crossing, etc.) is significantly different from a baseline PAP cycle duration. As another example, in some embodiments, AF is indicated if the durations of a relatively large number of PAP cycles differ by at least a specified amount.

A baseline PAP cycle duration may be defined in various ways. In some embodiments, the baseline is based on a prior PAP cycle duration. In some embodiments, the baseline is based on several prior PAP cycle durations (e.g., an average, mean, minimum, etc., of the prior PAP cycle durations). In some embodiments, the baseline is based on a defined PAP cycle duration (e.g., determined by simulations, empirical studies, or estimates).

Regarding amplitude parameters, irregularities in the ventricular cycles will lead to inefficient pumping by the cardiac chambers. For example, a shorter cardiac cycle may result in less blood than normal being pumped out of the heart during that cycle. Consequently, a subsequent cycle (e.g., the next cycle) may result in more blood than normal being pumped out of the heart. These changes in the amount of blood being moved during different cardiac cycles are manifested by a change in the amplitude (e.g., peak-to-peak amplitude) of the corresponding PAP waveform. Thus, AF may be indicated if the amplitude of one or more PAP cycles is significantly different from a baseline PAP cycle amplitude or if the amplitudes of a relatively large number of PAP cycles differ by at least a specified amount.

A baseline PAP cycle amplitude may be defined in various ways (e.g., in a similar manner as discussed above for the baseline PAP cycle duration). Thus, in various embodiments, the baseline may be based on a prior PAP cycle amplitude, several prior PAP cycle amplitudes, a defined PAP cycle amplitude, and so on.

Regarding morphology correlation parameters, irregularities in the ventricular cycles will lead to changes in the shape of the morphology for those cycles. For example, a shorter cardiac cycle may result in a compressed PAP morphology that may not correlate well with the morphology of other PAP cycles. Thus, AF may be indicated if the morphology of one or more PAP cycles is significantly different from (e.g., does not correlate well with) a baseline PAP cycle morphology or if the morphologies of a relatively large number of PAP cycles differ by at least a specified amount.

A baseline PAP cycle morphology may be defined in various ways (e.g., in a similar manner as discussed above for the baseline PAP cycle duration). Thus, in various embodiments, the baseline may be based on a prior PAP cycle morphology, several prior PAP cycle morphologies, a defined PAP cycle morphology, or so on.

Regarding frequency parameters, irregularities in the ventricular cycles will lead to changes in the frequency spectra for those cycles. For example, a shorter cardiac cycle may result in a wider frequency spectrum than other PAP cycles. Thus, AF may be indicated if the frequency information of one or more PAP cycles is significantly different from a baseline PAP cycle frequency information or if the frequency information of a relatively large number of PAP cycles differ by at least a specified amount.

Baseline PAP cycle frequency information may be defined in various ways (e.g., in a similar manner as discussed above for the baseline PAP cycle duration). Thus, in various embodiments, the baseline may be based on prior PAP cycle frequency information, frequency information of several prior PAP cycle morphologies, defined PAP cycle frequency information, and so on.

In a typical implementation, one stability parameter is determined for each parameter type employed at block 204. Thus, one stability parameter may be determined based on the set of timing parameters derived from the PAP data, another stability parameter may be determined based on the set of amplitude parameters derived from the PAP data, and so on.

A stability parameter may take various forms and may be calculated in various ways. For example, in various embodiments a stability parameter may comprise a variance, a standard deviation, a maximum variation (e.g., the difference between the parameter having the highest value and the parameter having the lowest value), an average variation, a mean variation, and so on.

As represented by block 208, the stability parameter(s) is/are compared with at least one threshold. In a typical embodiment, each stability parameter is compared with a corresponding threshold. For example, a timing-based stability parameter (e.g., the variance of the durations of the PAP cycles) may be compared to a timing-based threshold (e.g., a variance greater than or equal to 0.125 seconds may indicate AF). An amplitude-based stability parameter (e.g., the variance of the peak-to-peak amplitudes of the PAP cycles) may be compared to an amplitude-based threshold (e.g., a variance greater than or equal to 15% may indicate AF). A morphology-based stability parameter (e.g., the correlation of the morphology the PAP cycles) may be compared to a correlation-based threshold (e.g., a correlation less than or equal to 0.85 may indicate AF). A frequency-based stability parameter (e.g., the variance of the frequency spectrum width of the PAP cycles) may be compared to a frequency-based threshold (e.g., a variance greater than or equal to 15% may indicate AF).

As represented by block 210, an indication of AF is generated based on the comparison of block 208. The operations of block 210 depend in some aspects on the number of PAP parameters employed.

In an implementation that employs a single type of PAP parameter, the AF indication may be generated based on the comparison of the stability parameter with the threshold (e.g., a cycle timing variance greater than or equal to 0.125 seconds may result in the generation of the AF indication).

In an implementation that employs two or more types of PAP parameters, the AF indication may be generated based on the results of the comparison of each stability parameter with its corresponding threshold. Here, a decision to generate the AF indication may depend on how many of the stability parameters met (or exceeded or fell below) their respective thresholds. In some embodiments, an indication of AF is generated only if all of the comparisons indicated the likelihood of AF. In some embodiments, an indication of AF is generated only if a majority of the comparisons indicated the likelihood of AF. In some embodiments, an indication of AF is generated only if a defined number of the comparisons indicated the likelihood of AF. In some embodiments, an indication of AF is generated only if a defined percentage of the comparisons indicated the likelihood of AF.

In some embodiments, stability parameters are assigned weights. In this way, parameters that are designated as being more important will be accorded more significance in the AF detection operation. A simple example follows. In this example, four stability parameters A, B, C, and D are initially generated at block 206. The stability parameters A, B, C, and D are assigned weights of relative importance such as, for example, 0.5, 0.1, 0.6, and 0.1, respectively. The weighted parameters (e.g., a combination of the weighted parameters such as 0.5A+0.1B+0.6C+0.1D) are then compared to one or more thresholds at block 208 to determine whether an AF indication should be generated at block 210.

Through the use of multiple types of PAP parameters, a more selective indication of AF may be provided. Consequently, the likelihood of generating false AF indications may be reduced.

The AF indication may take different forms in different embodiments. In some embodiments, generating an indication involves setting a value in a memory device (e.g., setting a flag, setting bit in a register, setting a parameter stored in a memory, etc.). In some embodiments, generating an indication involves sending a signal (e.g., sending a signal (e.g., in the form of a message) to another device, transmitting an RF signal to another device, etc.). In some embodiments, generating an indication involves causing an indication to be output by a user interface circuit (e.g., a visual indication on a display screen, an audible indication output by a transducer, etc.).

In some embodiments, an ultimate indication of AF may be generated based on a PAP-derived AF indication and an AF indication derived in some other manner. For example, in a system where IEGM data is available, AF may be detected based on analysis of the IEGM data (e.g., based on the variation of the durations of the cardiac cycles). In such a case, an ultimate indication of AF may be generated only if AF is indicated by both the PAP-based AF detection and the IEGM-based AF detection.

As discussed herein, the operations of FIG. 2 will be repeated at a later point in time (e.g., periodically or based on a trigger). In some embodiments, one or more of the thresholds used by these operations may be dynamically adapted over time (e.g., based on physician feedback or some other criteria). Thus, a subsequent iteration of these operations may used a different threshold than a prior iteration of these operations. For example, a training feature may be employed whereby if it is determined (e.g., by an attending physician) that an episode has erroneously been classified as AF, information indicative of this determination may be fed back to the apparatus that performs the operations of FIG. 2. Upon receiving this information, one or more of the thresholds may then be adjusted (e.g., to reduce the sensitivity of the AF detection). In this way, the performance of the AF detection operation may be improved over time. It should be appreciated that the use of such a training feature also may result in a threshold being adjusted to increase the sensitivity of the AF detection in some cases.

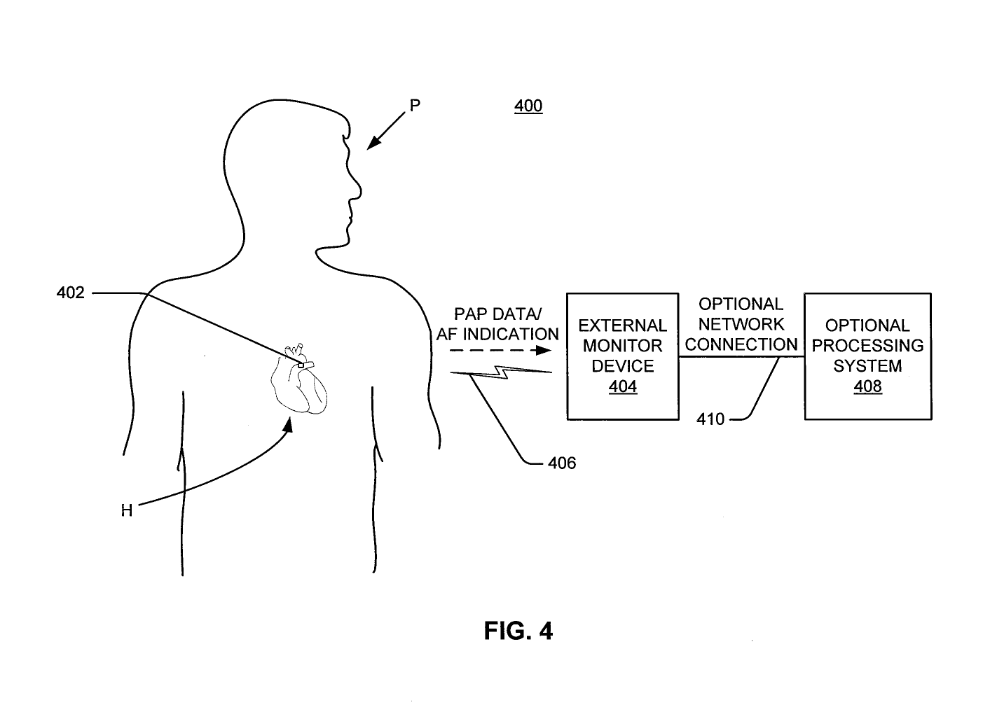

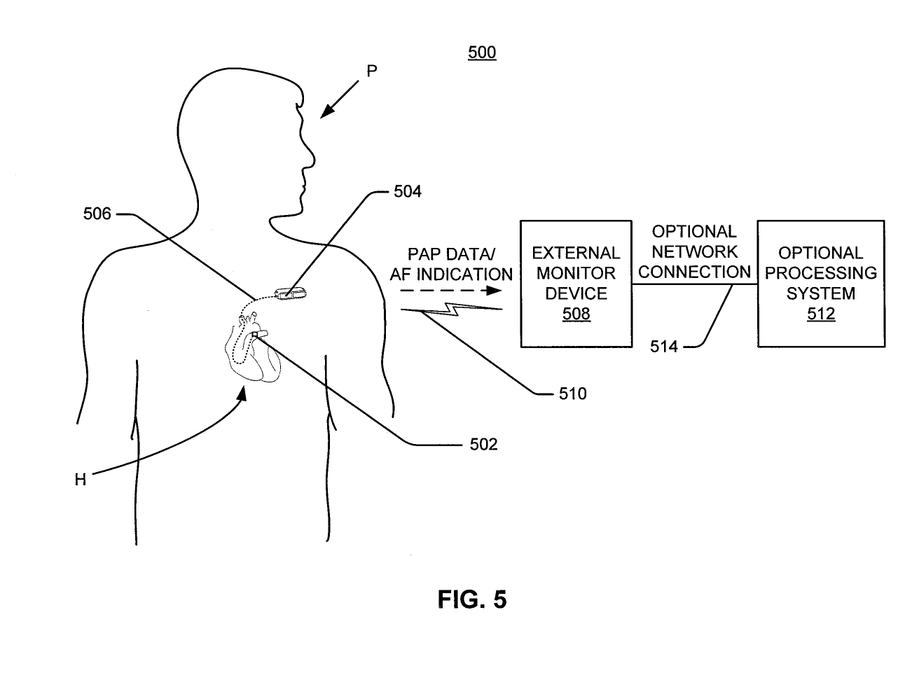

As mentioned above, PAP data may be transmitted to an external monitor device via an implantable PAP sensor device or via another implantable device. FIGS. 4 and 5 illustrate examples of the former and latter scenarios, respectively.

FIG. 4 is a simplified illustration of a communication system 400 where a PAP sensor device 402 implanted in the heart H of a patient P communicates with a monitor device 404 that is located external to the patient P. The PAP sensor device 402 and the monitor device 404 communicate with one another via a wireless communication link as represented by the symbol 406.

The monitor device 404 may take various forms. For example, the monitor device 404 may be a base station, a programmer, a home safety monitor, a personal monitor, a follow-up monitor, a wearable monitor, or some other type of device that is configured to communicate with the PAP sensor device 402.

The communication link 406 may be used to transfer information between the devices 402 and 404 in conjunction with various applications such as remote home-monitoring, clinical visits, data acquisition, remote follow-up, and portable or wearable patient monitoring/control systems. When information needs to be transferred between the devices 402 and 404, the patient P moves into a position that is relatively close to the monitor device 404, or vice versa. For example, the patient may sit, stand, or lie adjacent an antenna (e.g., a relatively large loop antenna) of the monitor device 404 to, depending on the embodiment, transfer PAP data or an AF indication from the PAP sensor device 402 to the monitor device 404 on a daily basis.

In some embodiments, the monitor device 404 communicates with another device. In the example of FIG. 4, this other device is represented by a processing system 408 that communicates with the external monitor device 404 via a network connection 410. The network connection 410 may take various forms including, for example, a local area network connection (e.g., Ethernet-based, Wi-Fi-based, Bluetooth-based, and so on), a wide area network connection (e.g., Internet-based, cellular-based, PSTN-based, and so on), a dedicated network connection, and so on.

In some embodiments, the monitor device 404 forwards information it receives from the PAP sensor device 402 to the processing system 408. For example, the monitor device 404 may forward PAP data or an AF indication in an unaltered form to the processing system 408. In some embodiments, the monitor device 404 sends information it generates to the processing system 408. For example, in embodiments where the monitor device 404 processes received PAP data to generate an AF indication, the monitor device 404 may send that AF indication to the processing system 408.

The processing system 408 may provide a more convenient means for a user (e.g., a clinician or a physician) to review patient information. For example, the processing system 408 may be a computer located at a user's office. As another example, the processing system 408 may be a server that is accessible by the user (e.g., via a computer, a tablet, a cell phone, etc.). In this way, the user may remotely access patient information (e.g., by accessing a website). The user may then review patient information at his or her convenience to determine whether medical intervention is warranted.

FIG. 5 is a simplified illustration of a communication system 400 where a PAP sensor device 502 implanted within a patient P is coupled with another implanted device 504. In the example of FIG. 5, the devices 502 and 504 are shown as being coupled via an implantable lead 506 (e.g., which may include one or more electrical conductors for transmitting communication signals and/or power). In other embodiments, the devices 502 and 504 may be coupled (e.g., for communication) via a wireless link. In embodiments where the devices 502 and 504 are configured to communicate with one another, the PAP sensor device 502 may send, depending on the embodiment, PAP data or an AF indication to the implanted device 504.

In the illustrated example, the implanted device 504 is an implantable cardiac device with one or more leads that are routed to the heart H of the patient P. For example, the implanted device 504 may be a pacemaker, an implantable cardioverter defibrillator, or some other similar device. It should be appreciated, however, that the implanted device 504 may take other forms.

The implanted device 504 communicates with a monitor device 508 that is located external to the patient P via a wireless communication link as represented by the symbol 510. In some embodiments, the implanted device 504 forwards information it receives from the PAP sensor device 402 to the monitor device 508. For example, the implanted device 504 may forward PAP data or an AF indication in an unaltered form to the monitor device 508. In some embodiments, the implanted device 504 sends information it generates to the monitor device 508. For example, in embodiments where the implanted device 504 processes received PAP data to generate an AF indication, the implanted device 504 may send the generated AF indication to the monitor device 508.

Operations of the monitor device 508 are generally similar to the operations of the monitor device 404 of FIG. 4. Thus, in some embodiments, the monitor device 508 communicates with another device (e.g., communicates with a processing system 512 via a network connection 514).

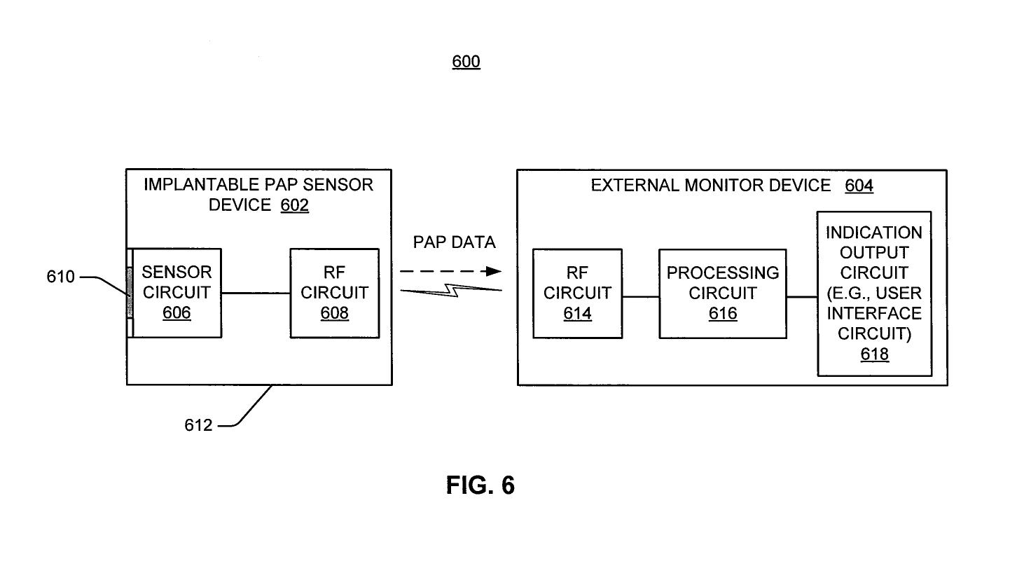

As mentioned above, the teachings herein may be implemented in different types of apparatuses. FIGS. 6-9 illustrate several examples of such apparatuses.

FIG. 6 illustrates an embodiment of a communication system 600 where an implantable PAP sensor device 602 sends PAP data to an external monitor device 604 that processes the PAP data to provide an AF indication. In particular, this figure depicts sample components of the PAP sensor device 602 and the monitor device 604 in such an embodiment.

The PAP sensor device 602 includes a sensor circuit 606 and an RF circuit 608. These circuits cooperate to provide PAP data to the external monitor device 604.

The sensor circuit 606 includes a flexible diaphragm 610 or some other suitable component that reacts (e.g., flexes) to changes in pressure external to the PAP sensor device 602. Upon implant in or near the pulmonary artery, the sensor circuit 606 is thus able to generate PAP data (e.g., analog signals or digital data) representative of the PAP as a result of the reaction of the flexible diaphragm 610.

The RF circuit 608 comprises circuitry that enables the monitor device 604 to acquire information from the PAP sensor device 602 via RF signaling. In some embodiments, the RF circuit 608 comprises a passive RF circuit with an antenna (e.g., as discussed below). In such a case, the PAP sensor device 602 need not have a source of internal power (e.g., a battery) since power may be obtained from received RF signals in this case. In some embodiments, the RF circuit 608 comprises an active RF circuit (e.g., an RF transceiver and associated antenna). In such a case, the PAP sensor device 602 will have a source of internal power (e.g., a battery) and/or circuitry to receive power from another device (e.g., as discussed below).

To facilitate implant, the PAP sensor device 602 is biocompatible and hermetically sealed. For example, the PAP pressure sensor 602 may include a biocompatible and hermetically sealed housing 612 (e.g., constructed of titanium or some other suitable material). In addition, the flexible diaphragm 610 may be coated with a biocompatible and hermetically sealed material (e.g., silicone) and attached to the housing 612 via an attachment technique that provides a hermetic seal.

In the example of FIG. 6, the external monitor device 604 includes circuitry for processing PAP data and generating an AF indication. An RF circuit 614 (e.g., an RF transceiver and an antenna) receives RF signals generated by the RF circuit 608 to acquire PAP data from the PAP sensor device 602. A processing circuit 616 processes the PAP data to generate the AF indication as taught herein. The AF indication is then output by an indication output circuit 618 (e.g., a user interface circuit such as a display device, an audio device, etc.). In this way, in the event a patient is experiencing AF while the PAP sensor device 602 is communicating with the external monitor device 604, the patient will be notified of this condition.

In a typical embodiment, the PAP sensor device 602 is of very small size to facilitate implant (e.g., via a venous approach). For example, the PAP sensor device 602 may be constructed using microelectromechanical system (MEMS) technology.

In some embodiments, the PAP sensor device 602 comprises a passive resonant inductor-capacitor circuit that is excited by an electromagnetic field generated by the external monitor device 604. In such a case, the sensor circuit 606 and RF circuit 608 comprise a single circuit: the resonant inductor-capacitor circuit. The capacitive circuit portion of the resonant circuit is flexible such that changes in pressure at the pressure sensor (e.g., as induced on a flexible material represented by the flexible diaphragm 610 of FIG. 6) cause changes in the capacitance of the capacitive circuit. Thus, changes in pressure at the PAP sensor device 602 (e.g., corresponding to the PAP waveform) are reflected by changes in the resonant frequency of the excited resonant circuit. These changes in the resonant frequency are then detected as signals (e.g., corresponding to a simple form of PAP data) by the RF circuit 614 of the external monitor device 604. The processing circuit 616 may then process the signals representative of the changes in the resonant frequency (e.g., after an analog-to-digital conversion process) to detect the changes in PAP and then provide data representative of a PAP waveform (e.g., as in FIG. 3). This data may then be processed as discussed herein for AF detection.

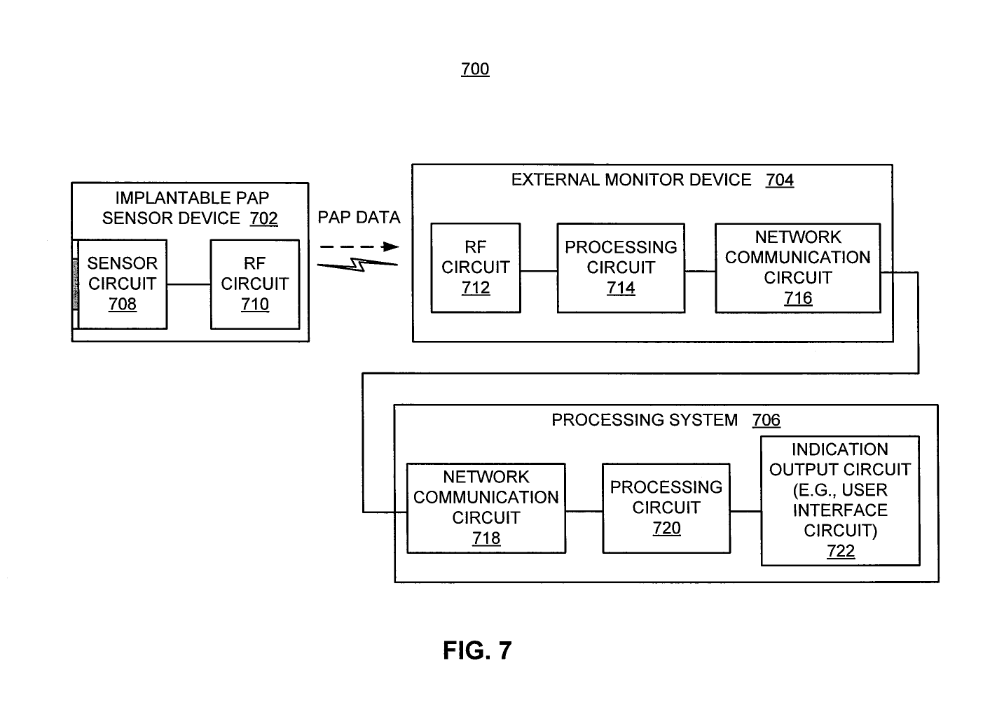

FIG. 7 illustrates an embodiment of a communication system 700 where an implantable PAP sensor device 702 sends PAP data to an external monitor device 704 which then sends information relating to (e.g., comprising or derived from) the PAP data to a processing system 706. Based on this information, the processing system 706 is able to provide an AF indication.

This PAP sensor device 702 includes a sensor circuit 708 and an RF circuit 710 to provide PAP data to the external monitor device 704. The operations of these components may be similar to the operations of similarly named components of FIG. 6.

In the example of FIG. 7, the external monitor device 704 may operate in one of two ways. In a first scenario, upon receiving the PAP data via the RF circuit 712, the processing circuit 714 will process the PAP data to generate an AF indication that is then sent to the processing system 706 via a network interface circuit 716. In a second scenario, upon receiving the PAP data via the RF circuit 712, the processing circuit 714 will simply forward the PAP data to the processing system 706 via the network interface circuit 716.

The processing system 706 includes a network communication circuit 718, a processing circuit 720, and an indication output circuit 618. These circuits may be configured in different ways depending on whether the processing system 706 receives PAP data or AF indications.

The network communication circuit 718 communicates with the network communication circuit 716 of the external monitor device 704. Thus, PAP data or an AF indication may be received from the external monitor device 704 via the network communication circuit 718.

Among other tasks, the processing circuit 720 processes information sent or received via the network communication circuit 718. Thus, in cases where PAP data is received by the processing system 706, the processing circuit 720 processes the PAP data to generate the AF indication as taught herein. The AF indication is then sent to the indication output circuit 722. In cases where AF indications are received by the processing system 706, the processing circuit 720 may simply forward each AF indication to the indication output circuit 722.

The indication output circuit 722 comprises a mechanism to provide an AF indication to one or more users. In some implementations, the indication output circuit 722 comprises a user interface circuit (e.g., a display device, an audio device, etc.). In some implementations, the indication output circuit 722 comprises a server (e.g., a web server) or some other suitable device that can be accessed by user devices (e.g., computers, laptops, cell phones, etc.) to provide an AF indication to the users via those user devices.

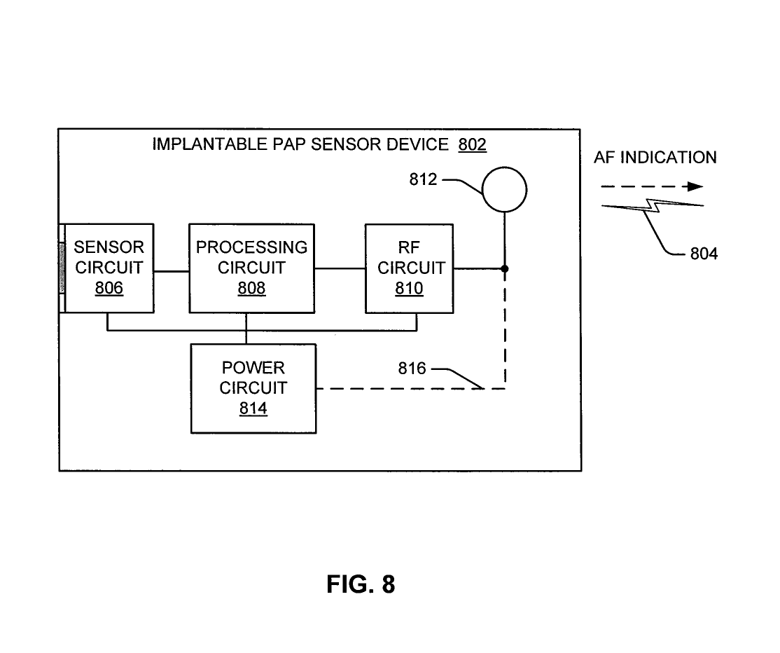

FIG. 8 illustrates an embodiment of an implantable PAP sensor device 802 that processes PAP data to provide an AF indication. In this example, the AF indication is transmitted wirelessly (e.g., to an external monitor device, not shown in FIG. 8) as represented by the symbol 804. The PAP sensor device 802 includes a sensor circuit 806 to provide PAP data (e.g., as discussed above at FIG. 6). A processing circuit 808 processes the PAP data to generate an AF indication as taught herein. An RF circuit 810 transmits the AF indication via an antenna 812 (e.g., a loop antenna).

A power circuit 814 provides power for the components of the PAP sensor device 802. In some embodiments, the power circuit comprises a battery.

In some embodiments, the power circuit 814 is coupled via a conductor 816 to the antenna 812 (or some other antenna, not shown) to receive electromagnetic energy from an external device (e.g., the external monitor device). In this case, the power circuit 814 uses the received electromagnetic energy to recharge a rechargeable battery or directly power the components of the PAP sensor device 802. For example, the power circuit 814 may include a rectifier that rectifies RF received signals, along with a power conditioning circuit that provides capacitive filtering or more robust filtering to generate a substantially DC rectified signal.

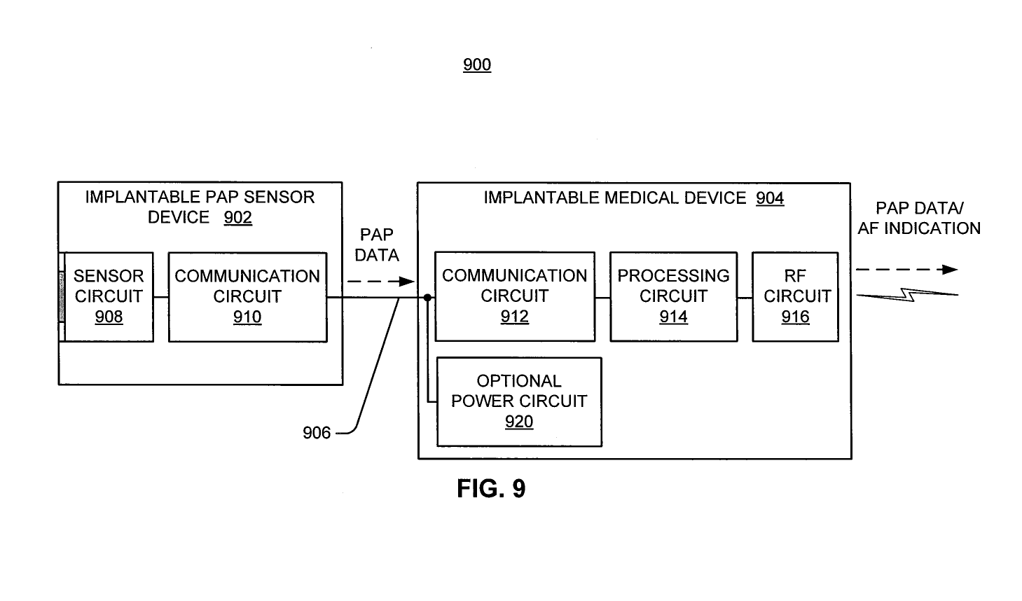

FIG. 9 illustrates an embodiment of a communication system 900 where an implantable PAP sensor device 902 is coupled to an implantable medical device 904 via an implantable lead 906. As discussed herein, the implantable medical device 904 may forward the PAP data or generate an AF indication based on the PAP data. For example, the implantable medical device 904 may transmit this information wirelessly to an external monitor device (not shown in FIG. 9).

The PAP sensor device 902 includes a sensor circuit 908 to provide PAP data (e.g., as discussed above at FIG. 6). In this case, however, the PAP data is provided to a communication circuit 910.

The communication circuit 910 sends the PAP data to the implantable medical device 904 via the implantable lead 906. For example, the communication circuit 910 may include processing circuitry for processing messages sent and received via the implantable lead 906. In addition, the communication circuit 910 may include driver and receiver components for driving signals and receiving signals on the implantable lead 906.

In the example of FIG. 9, the implantable medical device 904 may operate in one of two ways. In a first scenario, upon receiving the PAP data via a communication circuit 912 (e.g., which may be similar to the communication circuit 910), a processing circuit 914 will process the PAP data to generate an AF indication that is then transmitted via an RF circuit 916 (e.g., as discussed herein). In a second scenario, upon receiving the PAP data via the communication circuit 912, the processing circuit 914 will simply forward the PAP data to the RF circuit 916 for transmission.

The implantable lead 906 includes one or more signal paths (e.g., electrical conductors) for sending information between the devices 902 and 904. In addition, the implantable lead 906 will include a flexible lead body that is biocompatible and hermetically sealed (e.g., formed of silicone, polyurethane, plastic, or some other suitable biocompatible material). The implantable lead 906 and the devices 902 and 904 also typically include connectors to enable the implantable lead 906 to be connected to the devices 902 and 904. These connection mechanisms are also constructed of a biocompatible material and provide a hermetic seal.

In some embodiments, the implantable lead 906 includes at least one conductor for coupling power from a power circuit 920 to the PAP sensor device 902. For example, the power circuit 920 may include a battery that supplies power for the communication circuit 910 and, optionally, other components of the PAP sensor device 902.

In the example of FIG. 9, the PAP sensor device 902 sends PAP data to the implantable medical device 904. In other embodiments (e.g., where the PAP sensor device 902 includes a processing circuit), the PAP sensor device 902 may send an AF indication to the implantable medical device 904. In such a case, the implantable medical device 904 may forward that AF indication to an external device. For example, upon receipt of an AF indication by the communication circuit 912, the processing circuit 914 may forward the AF indication to the RF circuit 916 for transmission.

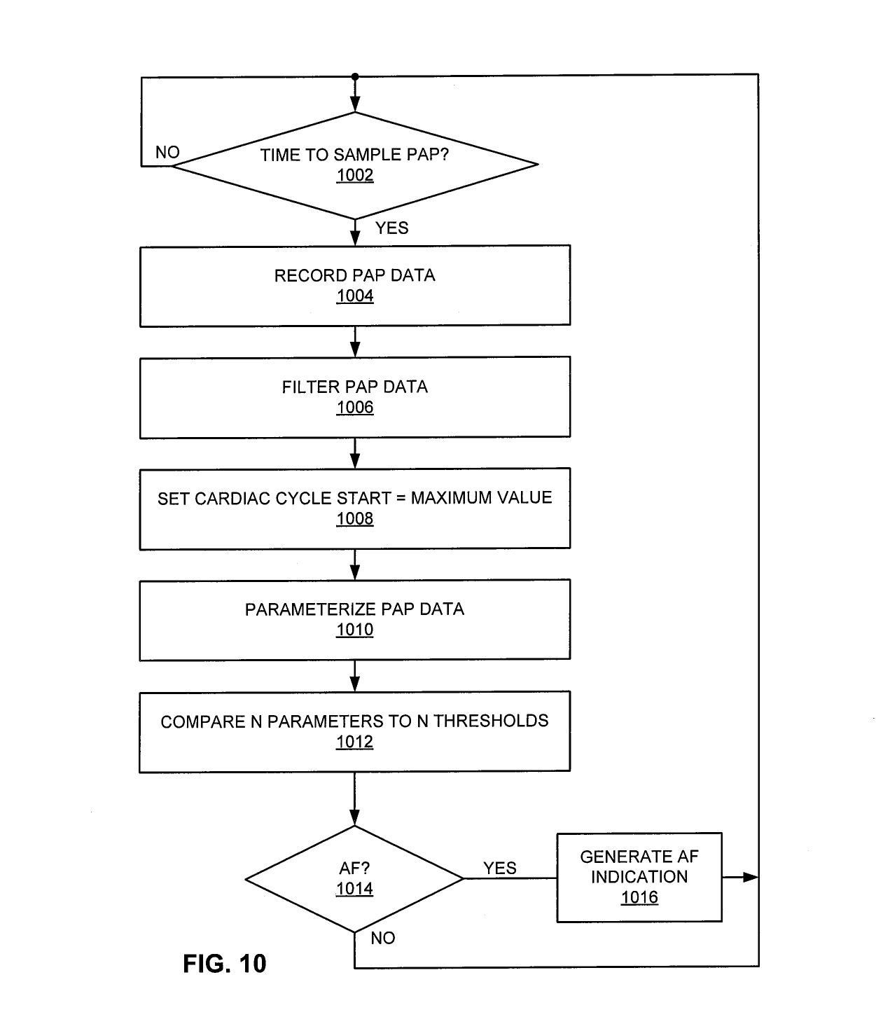

FIG. 10 illustrates additional operational details of a method for generating an indication of AF. As discussed herein, these operations may be performed by an implantable PAP sensor device, some other implantable device (e.g., an IMD), an external monitor device, or some other external device.

As represented by block 1002, at a programmable interval the PAP data acquisitions take place. This operation may be automated or patient managed (e.g., a patient initiates the measurements according to a schedule provided by a managing physician). Depending on the patient's unique situation, this interval may be varied substantially. One example would be to make the recordings once a day.

As represented by block 1004, at each data acquisition occasion, a plurality of cardiac cycles is recorded. For example, 30-40 seconds worth of data may be stored in a data memory device for each iteration of the process.

As represented by block 1006, the acquired data are filtered. For example, a band-pass filter may be employed to remove both high-frequency measurement noise and low-frequency noise due to respiration artifacts. As one example, such a filter may have a band-pass range on the order of 0.75 Hz to 30 Hz. After filtering, the PAP data may represent a PAP waveform such as the waveform 302 of FIG. 3.

As represented by block 1008, in embodiments where IEGM-based information is not available, the start of each cycle may be identified based on the maximum values of the PAP waveform. Here, upon identifying the maximum values, a marker may be placed there to identify a point of reference for that particular cardiac cycle.

Other techniques for identifying the start of a cycle may be employed in other embodiments. For example, autocorrelation functions and/or wavelet analysis may be employed to identify the start of each cardiac cycle.

As represented by block 1010, the PAP data is parameterized. This parameterization may be performed in various ways. Preferably, at least two parameterizations are used to increase the performance of the AF detector. Three examples of parameterizations include timing, amplitude, and morphology correlation as discussed herein.

For timing parameterization, the time between each point of reference may be calculated. The variance of these times then is calculated.

Peak-to-peak values may be used for amplitude parameterization. That is, the largest signal span may be determined for each cycle, and the variance of these values calculated.

For morphology correlation parameterization, a continuous sequence of the waveform (e.g., one heart cycle, or a longer or shorter sequence) is extracted. Correlations of all of these sequences extracted from the latest recorded session are then created and compared to each other. The variance of these correlations is then calculated.

As represented by block 1012, the parameters (e.g., N parameters) are compared to corresponding preset thresholds. These presets may be derived from real data and empirically fine tuned. In some aspects, these parameterizations may be chosen so that they will produce a large difference between values recorded during sinus rhythm versus during AF. In one embodiment, if a majority of these parameter values are above the preset thresholds (e.g., 2 out of 3), then the flag for AF detected is raised at block 1014.

As represented by block 1016, an AF indication is generated if the flag is raised. For example, the AF state may be communicated to the patient, so that the patient may be instructed to see his or her physician for follow-up. In this way, the risk of stroke may be acted upon.

If AF is not indicated at block 1014, the process returns to block 1002 and the above operations are repeated at some later point in time.

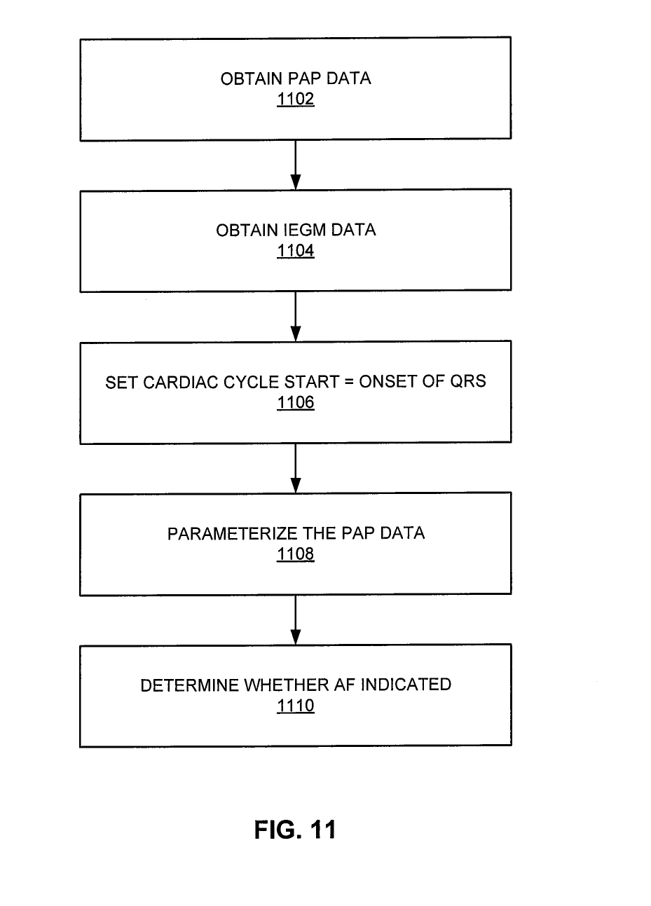

FIG. 11 illustrates an embodiment of operations that may be employed when IEGM-based information is available. For example, in the event PAP-based AF detection is to be used as an add-on to existing IMD-based AF detection, the system could be set up in such a way that if the IMD detects AF, then the above-described procedures may be activated to confirm or reject the IEGM-based AF adjudication. This joint output should increase (sensitivity and) specificity as compared to a single source of information. Moreover, if an IEGM signal is available, the QRS detections may be used to identify the start and end of each cardiac cycle in the PAP waveform.

Typically, the operations of FIG. 11 are preformed at designated intervals (e.g., as described above at block 1002 of FIG. 10). As represented by block 1102 of FIG. 11, PAP data is obtained (e.g., as discussed above). For example, an IMD may receive the PAP data from a PAP sensor device as discussed herein. As represented by block 1104, IEGM data is obtained. For example, data representative of a waveform such as the waveform 304 of FIG. 3 that was previously stored by the IMD may be retrieved by at this point. As represented by block 1106, the start of each cycle may be identified based on the QRS events indicated by the IEGM waveform. As represented by block 1108, this IEGM-derived cycle timing is then used during the parameterization of the PAP data. In particular, this timing information may be used to identify the time interval during which the cycle peak-to-peak amplitude is to be searched or during which the morphology information is to be collected for correlation operations. As represented by block 1110, a decision as to whether AF is indicated may then be made based on the parameters obtained at block 1108.

It should be appreciated that various modifications may be incorporated into the disclosed embodiments based on the teachings herein. For example, the structure and functionality taught herein may be incorporated into types of apparatuses other than the specific types of apparatuses described above.

Different embodiments of such an apparatus may include a variety of hardware and software processing components. In some embodiments, hardware components such as processors, controllers, state machines, logic, or some combination of these components, may be used to implement the described components or circuits (e.g., the processing circuits).

In some embodiments, code including instructions (e.g., software, firmware, middleware, etc.) may be executed on one or more processing devices (e.g., the processing circuits) to implement one or more of the described functions or components. The code and associated components (e.g., data structures and other components used by the code or used to execute the code) may be stored in an appropriate data memory that is readable by a processing device (e.g., commonly referred to as a computer-readable medium).

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.”

As used herein, the term “determining” encompasses a wide variety of actions. For example, “determining” may include calculating, computing, processing, deriving, investigating, looking up (e.g., looking up in a table, a database or another data structure), ascertaining, and the like. Also, “determining” may include receiving (e.g., receiving information), accessing (e.g., accessing data in a memory), and the like. Also, “determining” may include resolving, selecting, choosing, establishing, and the like.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. An apparatus for detecting atrial fibrillation, comprising:
a processing circuit configured to:
process pulmonary artery pressure data to determine pulmonary artery pressure parameters of a first type corresponding to a plurality of cardiac cycles,
determine a stability parameter indicative of variation of the pulmonary artery pressure parameters of the first type over the cardiac cycles,
compare the stability parameter to a threshold, and
generate an indication of atrial fibrillation based on the comparison.

2. The apparatus of claim 1, wherein the processing circuit is further configured to:
process the pulmonary artery pressure data to determine pulmonary artery pressure parameters of at least one second type corresponding to the cardiac cycles;
determine at least one other stability parameter indicative of variation of the pulmonary artery pressure parameters of the at least one second type over the cardiac cycles; and
compare the at least one other stability parameter to at least one other threshold, wherein the generation of the indication of atrial fibrillation is further based on the comparison of the at least one other stability parameter to the at least one other threshold.

3. The apparatus of claim 2, wherein the pulmonary artery pressure parameters of the first type and the at least one second type comprise at least two of the group consisting of: cycle-to-cycle timing parameters, per-cycle amplitude parameters, per-cycle morphology correlation parameters, and per-cycle frequency parameters.

4. The apparatus of claim 2, wherein:
the stability parameter and the at least one other stability parameter comprise at least three stability parameters;
the threshold and the at least one other threshold comprise at least three thresholds; and
the generation of the indication of atrial fibrillation is further based on whether a majority of the at least three stability parameters are greater than or equal to corresponding ones of the at least three thresholds.

5. The apparatus of claim 2, wherein:
the stability parameter and the at least one other stability parameter comprise at least three stability parameters;
the threshold and the at least one other threshold comprise at least three thresholds; and
the generation of the indication of atrial fibrillation is further based on whether a defined quantity of the at least three stability parameters are greater than or equal to corresponding ones of the at least three thresholds.

6. The apparatus of claim 2, wherein:
the stability parameter and the at least one other stability parameter comprise at least three stability parameters;
the threshold and the at least one other threshold comprise at least three thresholds; and
the generation of the indication of atrial fibrillation is further based on whether a defined percentage of the at least three stability parameters are greater than or equal to corresponding ones of the at least three thresholds.

7. The apparatus of claim 1, wherein the variation comprises a variance or a standard deviation.

8. The apparatus of claim 1, further comprising:
obtaining a second indication of atrial fibrillation based on intracardiac electrogram data; and
generating an ultimate indication of atrial fibrillation according to the second indication of atrial fibrillation and the indication of atrial fibrillation that is based on the comparison.

9. The apparatus of claim 1, wherein:
the apparatus is an implantable sensor device;
the apparatus further comprises a sensor circuit configured to measure pulmonary artery pressure to generate the pulmonary artery pressure data; and
the apparatus further comprises a radiofrequency circuit configured to transmit the indication of atrial fibrillation to a monitor device.

10. The apparatus of claim 1, wherein:
the apparatus is an implantable sensor device;
the apparatus further comprises a sensor circuit configured to measure pulmonary artery pressure to generate the pulmonary artery pressure data; and
the apparatus further comprises a communication circuit configured to send the indication of atrial fibrillation to an implantable cardiac device.

11. The apparatus of claim 1, wherein:
the apparatus is an implantable cardiac device;
the apparatus further comprises a communication circuit configured to receive the pulmonary artery pressure data from an implantable sensor device; and
the apparatus further comprises a radiofrequency circuit configured to transmit the indication of atrial fibrillation to a monitor device.

12. The apparatus of claim 1, wherein:
the apparatus is a monitor device;
the apparatus further comprises a radiofrequency circuit configured to receive the pulmonary artery pressure data from an implantable device;
the apparatus further comprises a user interface circuit; and
the processing circuit is further configured to send the indication of atrial fibrillation to the user interface circuit.

13. The apparatus of claim 1, wherein:
the apparatus is a monitor device;
the apparatus further comprises a radiofrequency circuit configured to receive the pulmonary artery pressure data from an implantable device; and
the apparatus further comprises a communication circuit configured to send the indication of atrial fibrillation to a processing system via a network connection.

14. The apparatus of claim 1, wherein:
the apparatus is a processing system;
the apparatus further comprises a communication circuit configured to receive the pulmonary artery pressure data via a network connection;
the apparatus further comprises a user interface circuit; and
the processing circuit is further configured to send the indication of atrial fibrillation to the user interface circuit.

15. An apparatus for detecting atrial fibrillation, comprising:
a processing circuit configured to:
process pulmonary artery pressure data to determine a plurality of different types of pulmonary artery pressure parameters corresponding to a plurality of cardiac cycles, wherein, for each pulmonary artery pressure parameter type, different pulmonary artery pressure parameters are determined for different cardiac cycles,
for each pulmonary artery pressure parameter type, determine a stability parameter indicative of variation of the pulmonary artery pressure parameters for the parameter type over the cardiac cycles,
compare the stability parameter to at least one threshold, and
generate an indication of atrial fibrillation based on the comparison of the stability parameters to the at least one threshold.

16. The apparatus of claim 15, wherein:
the processing circuit is further configured to assign weights to the stability parameters; and
the generation of the indication of atrial fibrillation is further based on the weighted stability parameters.

17. The apparatus of claim 16, wherein the plurality of different types of pulmonary artery pressure parameters comprise at least two of the group consisting of: cycle-to-cycle timing parameters, per-cycle amplitude parameters, per-cycle morphology correlation parameters, and per-cycle frequency parameters.

18. The apparatus of claim 17, wherein each variation comprises a variance or a standard deviation.

19. A method for detecting atrial fibrillation, comprising:
processing pulmonary artery pressure data to determine pulmonary artery pressure parameters of a first type corresponding to a plurality of cardiac cycles;
determining a stability parameter indicative of variation of the pulmonary artery pressure parameters of the first type over the cardiac cycles;
comparing the stability parameter to a threshold; and
generating an indication of atrial fibrillation based on the comparison.

20. The method of claim 19, further comprising:
processing the pulmonary artery pressure data to determine pulmonary artery pressure parameters of at least one second type corresponding to the cardiac cycles;
determining at least one other stability parameter indicative of variation of the pulmonary artery pressure parameters of the at least one second type over the cardiac cycles; and
comparing the at least one other stability parameter to at least one other threshold, wherein the generation of the indication of atrial fibrillation is further based on the comparison of the at least one other stability parameter to the at least one other threshold.
